(19) United States
(12) Patent Application Publication
(10) Pub. No.: US 2026/0275404 A1
(43) Pub. Date: Sep. 17, 2026

(54) METHODS FOR DUPLEX SEQUENCING

(71) Applicant: AccuraGen Holdings Limited, Georgetown (KY)

(72) Inventor: Li WENG, San Jose, CA (US)

(21) Appl. No.: 19/168,713

(22) PCT Filed: Mar. 26, 2024
(86) PCT No.: PCT/US2024/021506

§ 371 Date: Sep. 24, 2025

Related U.S. Application Data
(60) Provisional application No. 63/492,719, filed on Mar. 28, 2023.

(51) Int. Cl. C12Q 1/6806; C12Q 1/6869

(52) U.S. Cl. CPC C12Q 1/6806; C12Q 1/6869

(57) ABSTRACT

Provided herein are methods of processing a nucleic acid comprising providing a reaction mixture comprising: a plurality of double stranded nucleic acid molecules, and a plurality of oligonucleotides, wherein at least one of the plurality of oligonucleotides comprises a 5′ end and a 3′ end that are capable of base pairing with each other to yield a stem loop structure in the reaction mixture; subjecting the reaction mixture to a ligation reaction to ligate a first oligonucleotide of the plurality of oligonucleotides to a 5′ end of a double stranded nucleic acid molecule of the plurality of double stranded nucleic acid molecules and a second oligonucleotide of the plurality of oligonucleotides to a 3′ end of the double stranded nucleic acid molecule of the plurality of double stranded nucleic acid molecules, thereby creating at least one nucleic acid molecule without an exposed 5′ or 3′ end; modifying at least one nucleotide of the nucleic acid molecule to create a mismatched base pair within the nucleic acid molecule to yield a modified nucleic acid molecule; subjecting the modified nucleic acid molecule or derivative thereof to amplification with a primer to create a linear product comprising one or more copies of the modified nucleic acid molecule; and sequencing the linear product or a derivative thereof to obtain a sequence of a first strand and a second strand of the double stranded nucleic acid molecule.

### CROSS REFERENCE

This application claims the benefit of U.S. Provisional Application No. 63/492,719, filed Mar. 28, 2023, which is incorporated herein by reference in its entirety.

### BACKGROUND

Deoxyribonucleic acid (DNA) mainly exists as a double stranded helix in mammalian cells. As such, much of the DNA in a mammalian DNA sample is double stranded, having two complementary strands. Methods of duplex sequencing use the complementary strands to confirm rare sequence variants found in the first strand by checking the second strand.

### SUMMARY

In an aspect, provided herein are methods of processing a nucleic acid comprising providing a reaction mixture comprising: a plurality of double stranded nucleic acid molecules, a plurality of oligonucleotides, wherein at least one of the plurality of oligonucleotides comprises a 5′ end and a 3′ end that are capable of base pairing with each other to yield a stem loop structure in the reaction mixture. In some cases, the method comprises subjecting the reaction mixture to a ligation reaction to ligate a first oligonucleotide of the plurality of oligonucleotides to a 5′ end of a double stranded nucleic acid molecule of the plurality of double stranded nucleic acid molecules and a second oligonucleotide of the plurality of oligonucleotides to a 3′ end of the double stranded nucleic acid molecule of the plurality of double stranded nucleic acid molecules, thereby creating at least one nucleic acid molecule without an exposed 5′ or 3′ end. In some cases, the method comprises subjecting the nucleic acid molecule or derivative thereof to amplification with a primer to create a linear product comprising one or more copies of the nucleic acid molecule. In some cases, the method comprises sequencing the linear product or a derivative thereof to obtain a sequence of a first strand and a second strand of the double stranded nucleic acid molecule. In some cases, the first oligonucleotide or the second oligonucleotide comprises a methylated nucleotide. In some cases, the double stranded nucleic acid molecule comprises a methylated nucleotide. In some cases, the first oligonucleotide or the second oligonucleotide comprises one or more methylated cytosines. In some cases, the methylated nucleotide comprises methylated cytosine or methylated adenine. In some cases, the polymerase is a strand displacing polymerase. In some cases, subsequent to ligation, modifying at least one nucleotide of the nucleic acid molecule to create a mismatched base pair within the nucleic acid molecule to yield a modified nucleic acid molecule. In some cases, the modifying comprises a chemical or enzymatic modification that converts a methylated cytosine to a uracil or an unmethylated cytosine to a uracil. In some cases, the amplification product is a concatemer. In some cases, the method further comprises processing the sequence to identify a sequence variant when a sequence difference is present in the sequence of the first strand and the sequence of the second strand. In some cases, the sequence variant comprises a single nucleotide variant, a fusion, an insertion, or a deletion. In some cases, the method further comprises processing the sequence to identify an epigenetic modification when an epigenetic difference is present in the sequence of the first strand and confirmed by the sequence of the second strand. In some cases, the primer is a random primer. In some cases, the primer is a target specific primer. In some cases, the primer binds to a sequence of the first oligonucleotide or the second oligonucleotide. In some cases, the primer binds to a sequence of the first strand or the second strand of the double stranded nucleic acid molecule. In some cases, the plurality of double stranded nucleic acid molecules comprises double stranded deoxyribonucleic acid, double stranded ribonucleic acid, or a combination thereof. In some cases, the plurality of double stranded nucleic acid molecules is derived from a cell-free nucleic acid sample. In some cases, sequencing comprises bringing the plurality of sheared polynucleotides or a derivative thereof in contact with a plurality of nucleotides in the presence of a polymerase to incorporate one or more nucleotides of the plurality of nucleotides into a growing strand complementary to a strand of the sheared polynucleotides or derivative thereof, and detecting one or more signals indicative of incorporation of the one or more nucleotides into the growing strand. In some cases, the sequencing comprises sequencing by ligation. In some cases, the plurality of double stranded nucleic acid molecules is derived from a bodily fluid. In some cases, the bodily fluid comprises urine, saliva, blood, serum, sweat, or plasma. In some cases, the plurality of double stranded nucleic acids is derived from a biological sample of a subject, which biological sample comprises blood, saliva, serum, plasma, urine, menstrual fluid, sweat, skin, bone marrow, lung tissue, liver tissue, pancreatic tissue, stomach tissue, intestinal tissue, kidney tissue, bladder tissue, brain tissue, spinal cord tissue, tongue tissue, larynx tissue, esophageal tissue, spleen tissue, thymus tissue, thyroid tissue, breast tissue, prostate tissue, testicular tissue, or a tumor thereof. In some cases, the plurality of double stranded nucleic acid molecules is derived from a tumor sample of a subject.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows an example method for sequencing a double stranded nucleic acid molecule.

FIG. 2 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In an aspect, provided herein are methods of processing a nucleic acid. In some embodiments, the method comprises providing a reaction mixture comprising a plurality of double stranded nucleic acid molecules and a plurality of oligonucleotides. In some embodiments, at least one of the plurality of oligonucleotides comprises a 5′ end and a 3′ end that are capable of base pairing with each other to yield a stem loop structure in the reaction mixture. Next, the method can comprise subjecting the reaction mixture to a ligation reaction to ligate a first oligonucleotide of the plurality of oligonucleotides to a 5′ end of a double stranded nucleic acid molecule of the plurality of double stranded nucleic acid molecules and a second oligonucleotide of the plurality of oligonucleotides to a 3′ end of the double stranded nucleic acid molecule of the plurality of double stranded nucleic acid molecules, thereby creating at least one nucleic acid molecule without an exposed 5′ or 3′ end. Next, the method can comprise subjecting the nucleic acid molecule or derivative thereof to amplification with a primer to create a linear product comprising one or more copies of the nucleic acid molecule. Then the method can comprise sequencing the linear product or a derivative thereof to obtain a sequence of a first strand and a second strand of the double stranded nucleic acid molecule.

In various aspects of methods provided herein, the double stranded nucleic acid molecule and/or the first oligonucleotide or the second oligonucleotide comprise an epigenetic mark. In some cases, the first oligonucleotide or the second oligonucleotide comprises a methylated nucleotide. Alternatively or in combination, the double stranded nucleic acid molecule comprises a methylated nucleotide. In some cases, the first oligonucleotide or the second oligonucleotide comprises one or more methylated cytosines. Alternatively or in combination, the methylated nucleotide comprises methylated cytosine or methylated adenine In various aspects of methods provided herein, the amplification step creates a linear product that comprises one or more copies of the nucleic acid molecule without an exposed 5′ or 3′ end. In some cases, the amplification step requires a polymerase that can continually make copies of this molecule. In some cases, the method uses a polymerase that is a strand displacing polymerase. In some cases, the method uses an amplification method and enzymes described elsewhere herein. In some cases, the amplification product is a concatemer. In some cases, the amplification step uses a primer that is a random primer. In some cases, the amplification step uses a primer that is a target specific primer. In some cases, the amplification step uses a primer that binds to a sequence of the first oligonucleotide or the second oligonucleotide. In some cases, the amplification step uses a primer that binds to a sequence of said first strand or second strand of said double stranded nucleic acid molecule.

In various aspects of methods provided herein, the method can comprise modifying at least one nucleotide of the nucleic acid molecule to create a mismatched base pair within the nucleic acid molecule to yield a modified nucleic acid molecule. In some cases, the modifying the modifying comprises a chemical or enzymatic modification. In some embodiments, the chemical or enzymatic modification converts a methylated cytosine to a uracil. In some cases, the chemical or enzymatic modification converts an unmethylated cytosine to a uracil. In some cases, the chemical modification comprises exposing the nucleic acid molecule to a nucleic acid damage agent. In some cases, the nucleic acid damage agent comprises a chemotherapy agent.

In various aspects of methods provided herein the sequencing can comprise sequencing by synthesis. In some embodiments, sequencing comprises bringing the plurality of sheared polynucleotides or a derivative thereof in contact with a plurality of nucleotides in the presence of a polymerase to incorporate one or more nucleotides of the plurality of nucleotides into a growing strand complementary to a strand of the sheared polynucleotides or derivative thereof and detecting one or more signals indicative of incorporation of the one or more nucleotides into the growing strand. In some cases, the sequencing comprises sequencing by ligation.

In various aspects of methods provided herein, the method can further comprise processing the sequence to identify a sequence variant. In some cases, the sequence variant is identified when a sequence difference is present in the sequence of the first strand and the sequence of the second strand. In some cases, the sequence variant comprises a single nucleotide variant, a fusion, an insertion, a deletion. In some cases, the method further comprises processing the sequence to identify an epigenetic modification. In some cases, the epigenetic variant is identified with the epigenetic modification is present in the sequence of the first strand and confirmed by the sequence of the second strand of the double stranded nucleic acid molecule. In particular cases, the epigenetic modification occurs only on the first strand, for example there when there is a methylated C on the first strand, there is a G on the second strand, and when bisulfite conversion is used to detect the methylated C, the first strand is converted to a T and the second strand continues to be a G, and the methylated C is inferred based on the occurrence of the G on the second strand.

In various aspects of methods provided herein the plurality of double stranded nucleic acid molecules can comprise double stranded deoxyribonucleic acid, double stranded ribonucleic acid, or a combination thereof.

In various aspects of methods provided herein, the plurality of double stranded nucleic acid molecules can be derived from a cell-free nucleic acid sample. In some cases, the plurality of double stranded nucleic acid molecules is derived from a bodily fluid. In some cases, the bodily fluid comprises urine, saliva, blood, serum, sweat, or plasma. In some cases, the plurality of double stranded nucleic acids is derived from a biological sample of a subject, which biological sample comprises blood, saliva, serum, plasma, urine, menstrual fluid, sweat, skin, bone marrow, lung tissue, liver tissue, pancreatic tissue, stomach tissue, intestinal tissue, kidney tissue, bladder tissue, brain tissue, spinal cord tissue, tongue tissue, larynx tissue, esophageal tissue, spleen tissue, thymus tissue, thyroid tissue, breast tissue, prostate tissue, testicular tissue, or a tumor thereof. In some cases, the plurality of double stranded nucleic acid molecules is derived from a tumor sample of a subject.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment may be assessed using any suitable parameters of a chosen algorithm, including default parameters.

In general, the term “sequence variant” refers to any variation in sequence relative to one or more reference sequences. Typically, the sequence variant occurs with a lower frequency than the reference sequence for a given population of individuals for whom the reference sequence is known. For example, a particular bacterial genus may have a consensus reference sequence for the 16S rRNA gene, but individual species within that genus may have one or more sequence variants within the gene (or a portion thereof) that are useful in identifying that species in a population of bacteria. As a further example, sequences for multiple individuals of the same species (or multiple sequencing reads for the same individual) may produce a consensus sequence when optimally aligned, and sequence variants with respect to that consensus may be used to identify mutants in the population indicative of dangerous contamination. In general, a “consensus sequence” refers to a nucleotide sequence that reflects the most common choice of base at each position in the sequence where the series of related nucleic acids has been subjected to intensive mathematical and/or sequence analysis, such as optimal sequence alignment according to any of a variety of sequence alignment algorithms. A variety of alignment algorithms are available, some of which are described herein. In some embodiments, the reference sequence is a single known reference sequence, such as the genomic sequence of a single individual. In some embodiments, the reference sequence is a consensus sequence formed by aligning multiple known sequences, such as the genomic sequence of multiple individuals serving as a reference population, or multiple sequencing reads of polynucleotides from the same individual. In some embodiments, the reference sequence is a consensus sequence formed by optimally aligning the sequences from a sample under analysis, such that a sequence variant represents a variation relative to corresponding sequences in the same sample. In some embodiments, the sequence variant occurs with a low frequency in the population (also referred to as a “rare” sequence variant). For example, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some embodiments, the sequence variant occurs with a frequency of about or less than about 0.1%.

A sequence variant can be any variation with respect to a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a sequence variant comprises two or more nucleotide differences, the nucleotides that are different may be contiguous with one another, or discontinuous. Non-limiting examples of types of sequence variants include single nucleotide polymorphisms (SNP), deletion/insertion polymorphisms (DIP), copy number variants (CNV), short tandem repeats (STR), simple sequence repeats (SSR), variable number of tandem repeats (VNTR), amplified fragment length polymorphisms (AFLP), retrotransposon-based insertion polymorphisms, sequence specific amplified polymorphism, and differences in epigenetic marks that can be detected as sequence variants (e.g. methylation differences).

Polynucleotides from a sample may be any of a variety of polynucleotides, including but not limited to, DNA, RNA, ribosomal RNA (rRNA), transfer RNA (tRNA), micro-RNA (miRNA), messenger RNA (mRNA), fragments of any of these, or combinations of any two or more of these. In some embodiments, samples comprise DNA. In some embodiments, samples comprise genomic DNA. In some embodiments, samples comprise mitochondrial DNA, chloroplast DNA, plasmid DNA, bacterial artificial chromosomes, yeast artificial chromosomes, oligonucleotide tags, or combinations thereof. In some embodiments, the samples comprise DNA generated by amplification, such as by primer extension reactions using any suitable combination of primers and a DNA polymerase, including but not limited to polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. In general, sample polynucleotides comprise any polynucleotide present in a sample, which may or may not include target polynucleotides. The polynucleotides may be single-stranded, double-stranded, or a combination of these. In some embodiments, polynucleotides subjected to a method of the disclosure are single-stranded polynucleotides, which may or may not be in the presence of double-stranded polynucleotides. In some embodiments, the polynucleotides are single-stranded DNA. Single-stranded DNA (ssDNA) may be ssDNA that is isolated in a single-stranded form, or DNA that is isolated in double-stranded form and subsequently made single-stranded for the purpose of one or more steps in a method of the disclosure.

If a sample is treated to extract polynucleotides, such as from cells in a sample, a variety of extraction methods are available. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see e.g., U.S. Pat. No. 5,705,628). In some embodiments, the above isolation methods may be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic acid isolation step, purification of nucleic acids can be performed after any step in the disclosed methods, such as to remove excess or unwanted reagents, reactants, or products. A variety of methods for determining the amount and/or purity of nucleic acids in a sample are available, such as by absorbance (e.g., absorbance of light at 260 nm, 280 nm, and a ratio of these) and detection of a label (e.g., fluorescent dyes and intercalating agents, such as SYBR green, SYBR blue, DAPI, propidium iodine, Hoechst stain, SYBR gold, ethidium bromide).

Where desired, polynucleotides from a sample may be fragmented prior to further processing. Fragmentation may be accomplished by any of a variety of methods, including chemical, enzymatic, and mechanical fragmentation. In some embodiments, the fragments have an average or median length from about 10 to about 1,000 nucleotides in length, such as between 10-800, 10-500, 50-500, 90-200, or 50-150 nucleotides. In some embodiments, the fragments have an average or median length of about or less than about 100, 200, 300, 500, 600, 800, 1000, or 1500 nucleotides. In some embodiments, the fragments range from about 90-200 nucleotides, and/or have an average length of about 150 nucleotides. In some embodiments, the fragmentation is accomplished mechanically comprising subjecting sample polynucleotides to acoustic sonication. In some embodiments, the fragmentation comprises treating the sample polynucleotides with one or more enzymes under conditions suitable for the one or more enzymes to generate double-stranded nucleic acid breaks. Examples of enzymes useful in the generation of polynucleotide fragments include sequence specific and non-sequence specific nucleases. Non-limiting examples of nucleases include DNase I, Fragmentase, restriction endonucleases, variants thereof, and combinations thereof. For example, digestion with DNase I can induce random double-stranded breaks in DNA in the absence of Mg++ and in the presence of Mn++. In some embodiments, fragmentation comprises treating the sample polynucleotides with one or more restriction endonucleases. Fragmentation can produce fragments having 5′ overhangs, 3′ overhangs, blunt ends, or a combination thereof. In some embodiments, such as when fragmentation comprises the use of one or more restriction endonucleases, cleavage of sample polynucleotides leaves overhangs having a predictable sequence. Fragmented polynucleotides may be subjected to a step of size selecting the fragments via standard methods such as column purification or isolation from an agarose gel.

Oligonucleotides ligated to said double stranded nucleic acid molecules can contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different oligonucleotides or subsets of different oligonucleotides, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as flow cells as developed by Illumina, Inc.), one or more random or near-random sequences (e.g. one or more nucleotides selected at random from a set of two or more different nucleotides at one or more positions, with each of the different nucleotides selected at one or more positions represented in a pool of oligonucleotides comprising the random sequence), and combinations thereof. In some embodiments, the oligonucleotides in the ligation product can also be used as a sequencing primer. Two or more sequence elements can be non-adjacent to one another (e.g., separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the oligonucleotide. A sequence element may be of any suitable length, such as about or less than about 3, 4, 5, 6, 7, 8, 9, 10, 15, 20, 25, 30, 35, 40, 45, 50 or more nucleotides in length.

Oligonucleotides can have any suitable length, at least sufficient to accommodate the one or more sequence elements of which they are comprised. In some embodiments, oligonucleotides are about or less than about 10, 15, 20, 25, 30, 35, 40, 45, 50, 55, 60, 65, 70, 75, 80, 90, 100, 200, or more nucleotides in length. In some embodiments, an oligonucleotide is in the range of about 12 to 40 nucleotides in length, such as about 15 to 35 nucleotides in length.

In some embodiments, the oligonucleotides joined to fragmented polynucleotides from one sample comprise one or more sequences common to all oligonucleotides and a barcode that is unique to the oligonucleotides joined to double stranded nucleic acid molecules of that particular sample, such that the barcode sequence can be used to distinguish polynucleotides originating from one sample or oligonucleotide joining reaction from polynucleotides originating from another sample or oligonucleotide joining reaction. In some embodiments, an oligonucleotide comprises a 5′ overhang, a 3′ overhang, or both that is complementary to one or more target polynucleotide overhangs. Complementary overhangs can be one or more nucleotides in length, including but not limited to 1, 2, 3, 4, 5, 6, 7, 8, 9, 10, 11, 12, 13, 14, 15, or more nucleotides in length. Complementary overhangs may comprise a fixed sequence. Complementary overhangs of an oligonucleotide may comprise a random sequence of one or more nucleotides, such that one or more nucleotides are selected at random from a set of two or more different nucleotides at one or more positions, with each of the different nucleotides selected at one or more positions represented in a pool of oligonucleotides with complementary overhangs comprising the random sequence. In some embodiments, an oligonucleotide overhang is complementary to a target polynucleotide overhang produced by restriction endonuclease digestion. In some embodiments, an oligonucleotide overhang consists of an adenine or a thymine.

A variety of methods for ligating a first oligonucleotide to a 5′ end of a double stranded nucleic acid molecule and a second oligonucleotide to a 3′ end of said double stranded nucleic acid molecule to create a nucleic acid molecule without an exposed 5′ or 3′ end is available. In some embodiments, ligation comprises an enzymatic reaction, such as use of a ligase (e.g., an RNA or DNA ligase). A variety of ligases are available, including, but not limited to, RNA ligase, T4 DNA ligase, Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof. In some embodiments, reaction times are between 12-16 hours. Reaction conditions may be those specified by the manufacturer of the selected enzyme. In some embodiments, an exonuclease step can be included to digest any unligated nucleic acids after the ligation reaction. That is, desired ligation products do not contain a free 5′ or 3′ end, and thus the introduction of a 5′ or 3′ exonuclease will not digest the ligation products but will digest the unligated components.

After ligation, reaction products may be purified prior to amplification or sequencing to increase the relative concentration or purity of nucleic acid molecules without an exposed 5′ or 3′ end available for participating in subsequence steps (e.g., by isolation of nucleic acid molecules without an exposed 5′ or 3′ end or removal of one or more other molecules in the reaction). For example, a ligation reaction or components thereof may be treated to remove nucleic acid molecules having an exposed 5′ or 3′ end, such as by treatment with an exonuclease. As a further example, a ligation reaction or portion thereof may be subjected to size exclusion chromatography, whereby small reagents are retained and discarded (e.g., unreacted adapters), or ligation products are retained and released in a separate volume. A variety of kits for cleaning up ligation reactions are available, such as kits provided by Zymo oligo purification kits made by Zymo Research. In some embodiments, purification comprises treatment to remove or degrade ligase used in the ligation reaction, and/or to purify circularized polynucleotides away from such ligase.

In some embodiments, treatment to degrade ligase comprises treatment with a protease, such as proteinase K, or a heat-labile protease. Proteinase K treatment may follow manufacturer protocols, or standard protocols (e.g., as provided in Sambrook and Green, Molecular Cloning: A Laboratory Manual, 4th Edition (2012)). Protease treatment may also be followed by extraction and precipitation. In one example, ligated nucleic acids are purified by proteinase K (Qiagen) treatment in the presence of 0.1% SDS and 20 mM EDTA, extracted with 1:1 phenol/chloroform and chloroform, and precipitated with ethanol or isopropanol. In some embodiments, precipitation is in ethanol. In some embodiments, when a heat-labile protease is used, extraction and precipitation is not necessary.

Ligation may be followed directly by sequencing the nucleic acid molecules without an exposed 5′ or 3′ end. Alternatively, sequencing may be preceded by one or more amplification reactions. In general, “amplification” refers to a process by which one or more copies are made of a target polynucleotide or a portion thereof. A variety of methods of amplifying polynucleotides (e.g., DNA and/or RNA) are available. Amplification may be linear, exponential, or involve both linear and exponential phases in a multi-phase amplification process. Amplification methods may involve changes in temperature, such as a heat denaturation step, or may be isothermal processes that do not require heat denaturation. Polymerase chain reaction (PCR) uses multiple cycles of denaturation, annealing of primer pairs to opposite strands, and primer extension to exponentially increase copy numbers of the target sequence. Denaturation of annealed nucleic acid strands may be achieved by the application of heat, increasing local metal ion concentrations (e.g., U.S. Pat. No. 6,277,605), ultrasound radiation (e.g., WO/2000/049176), application of voltage (e.g., U.S. Pat. Nos. 5,527,670, 6,033,850, 5,939,291, and 6,333,157), and application of an electromagnetic field in combination with primers bound to a magnetically-responsive material (e.g., U.S. Pat. No. 5,545,540). In a variation called RT-PCR, reverse transcriptase (RT) is used to make a complementary DNA (cDNA) from RNA, and the cDNA is then amplified by PCR to produce multiple copies of DNA (e.g., U.S. Pat. Nos. 5,322,770 and 5,310,652). One example of an isothermal amplification method is strand displacement amplification, commonly referred to as SDA, which uses cycles of annealing pairs of primer sequences to opposite strands of a target sequence, primer extension in the presence of a dNTP to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product (e.g. U.S. Pat. Nos. 5,270,184 and 5,455,166). Thermophilic SDA (tSDA) uses thermophilic endonucleases and polymerases at higher temperatures in essentially the same method (European Pat. No. 0 684 315).

Other amplification methods include rolling circle amplification (RCA) (e.g., Lizardi, “Rolling Circle Replication Reporter Systems,” U.S. Pat. No. 5,854,033); helicase dependent amplification (HDA) (e.g., Kong et al., “Helicase Dependent Amplification Nucleic Acids,” U.S. Pat. Appln. Pub. No. US 2004-0058378 A1); and loop-mediated isothermal amplification (LAMP) (e.g., Notomi et al., “Process for Synthesizing Nucleic Acid,” U.S. Pat. No. 6,410,278). In some cases, isothermal amplification utilizes transcription by an RNA polymerase from a promoter sequence, such as may be incorporated into an oligonucleotide primer. Transcription-based amplification methods include nucleic acid sequence-based amplification, also referred to as NASBA (e.g., U.S. Pat. No. 5,130,238); methods which rely on the use of an RNA replicase to amplify the probe molecule itself, commonly referred to as QB replicase (e.g., Lizardi, P. et al. (1988) BioTechnol. 6, 1197-1202); self-sustained sequence replication (e.g., Guatelli, J. et al. (1990) Proc. Natl. Acad. Sci. USA 87, 1874-1878; Landgren (1993) Trends in Genetics 9, 199-202; and HELEN H. LEE et al., NUCLEIC ACID AMPLIFICATION TECHNOLOGIES (1997)); and methods for generating additional transcription templates (e.g., U.S. Pat. Nos. 5,480,784 and 5,399,491). Further methods of isothermal nucleic acid amplification include the use of primers containing non-canonical nucleotides (e.g., uracil or RNA nucleotides) in combination with an enzyme that cleaves nucleic acids at the non-canonical nucleotides (e.g., DNA glycosylase or RNaseH) to expose binding sites for additional primers (e.g., U.S. Pat. Nos. 6,251,639, 6,946,251, and 7,824,890). Isothermal amplification processes can be linear or exponential.

In some embodiments, amplification comprises rolling circle amplification (RCA). A typical RCA reaction mixture comprises one or more primers, a polymerase, and dNTPs, and produces concatemers. Typically, the polymerase in an RCA reaction is a polymerase having strand-displacement activity. A variety of such polymerases are available, non-limiting examples of which include exonuclease minus DNA Polymerase I large (Klenow) Fragment, Phi29 DNA polymerase, Taq DNA Polymerase, and the like. In general, a concatemer is a polynucleotide amplification product comprising two or more copies of a target sequence from a template polynucleotide (e.g., more than about 2, 3, 4, 5, 6, 7, 8, 9, 10, or more copies of the target sequence; in some embodiments, about or more than about 2 copies). Amplification primers may be of any suitable length, such as about or at least about 5, 10, 15, 20, 25, 30, 35, 40, 45, 50, 55, 60, 65, 70, 75, 80, 90, 100, or more nucleotides, any portion or all of which may be complementary to the corresponding target sequence to which the primer hybridizes (e.g. about, or at least about 5, 10, 15, 20, 25, 30, 35, 40, 45, 50, or more nucleotides). In some embodiments, multiple target-specific primers for a plurality of targets are used in the same reaction. For example, target-specific primers for about or at least about 10, 50, 100, 150, 200, 250, 300, 400, 500, 1000, 2500, 5000, 10000, 15000, or more different target sequences may be used in a single amplification reaction in order to amplify a corresponding number of target sequences (if present) in parallel. Multiple target sequences may correspond to different portions of the same gene, different genes, or non-gene sequences. Where multiple primers target multiple target sequences in a single gene, primers may be spaced along the gene sequence (e.g., spaced apart by about or at least about 50 nucleotides, every 50-150 nucleotides, or every 50-100 nucleotides) in order to cover all or a specified portion of a target gene.

In some embodiments, amplification is done with random primers. In general, a random primer comprises one or more random or near-random sequences (e.g., one or more nucleotides selected at random from a set of two or more different nucleotides at one or more positions, with each of the different nucleotides selected at one or more positions represented in a pool of adapters comprising the random sequence). In this way, polynucleotides (e.g., all, or substantially all polynucleotides) can be amplified in a sequence non-specific fashion. Such procedures may be referred to as “whole genome amplification” (WGA); however, typical WGA protocols (which do not involve a ligation step) do not efficiently amplify short polynucleotides, such as polynucleotide fragments contemplated by the present disclosure. For further illustrative discussion of WGA procedures, see for example Li et al (2006) J Mol. Diagn. 8(1): 22-30.

Where nucleic acid molecules without an exposed 5′ or 3′ end are amplified prior to sequencing, amplified products may be subjected to sequencing directly without enrichment, or subsequent to one or more enrichment steps. Enrichment may comprise purifying one or more reaction components, such as by retention of amplification products or removal of one or more reagents. For example, amplification products may be purified by hybridization to a plurality of probes attached to a substrate, followed by release of captured polynucleotides, such as by a washing step. Alternatively, amplification products can be labeled with a member of a binding pair followed by binding to the other member of the binding pair attached to a substrate and washing to release the amplification product. Possible substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica, or silica-based materials including silicon and modified silicon, carbon, metals, inorganic glasses, plastics, optical fiber bundles, and a variety of other polymers. In some embodiments, the substrate is in the form of a bead or other small, discrete particle, which may be a magnetic or paramagnetic bead to facilitate isolation through application of a magnetic field. In general, “binding pair” refers to one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine); biotin/avidin (or biotin/streptavidin); calmodulin binding protein (CBP)/calmodulin; hormone/hormone receptor; lectin/carbohydrate; peptide/cell membrane receptor; protein A/antibody; hapten/antihapten; enzyme/cofactor; and enzyme/substrate.

According to some embodiments, nucleic acid molecules without an exposed 5′ or 3′ end (or amplification products thereof, which may have optionally been enriched) are subjected to a sequencing reaction to generate sequencing reads. Sequencing reads produced by such methods may be used in accordance with other methods disclosed herein. A variety of sequencing methodologies are available, particularly high-throughput sequencing methodologies. Examples include, without limitation, sequencing systems manufactured by Illumina (sequencing systems such as HiSeq® and MiSeq®), Life Technologies (Ion Torrent®, SOLID®, etc.), Roche's 454 Life Sciences systems, Pacific Biosciences systems, etc. In some embodiments, sequencing comprises use of HiSeq® and MiSeq® systems to produce reads of about or more than about 50, 75, 100, 125, 150, 175, 200, 250, 300, or more nucleotides in length. In some embodiments, sequencing comprises a sequencing by synthesis process, where individual nucleotides are identified iteratively, as they are added to the growing primer extension product. Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the a and B phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891.

In related sequencing processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. For example, immobilization of the complex can be via a linkage between the polymerase or the primer and the substrate surface. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex, allowing identification of the incorporated nucleotide. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various nucleotides are cycled through the reaction mixture in the same process. See, e.g., U.S. Pat. No. 6,833,246, incorporated herein by reference in its entirety for all purposes. For example, the Illumina Genome Analyzer System is based on technology described in WO 98/44151, wherein DNA molecules are bound to a sequencing platform (flow cell) via an anchor probe binding site (otherwise referred to as a flow cell binding site) and amplified in situ on a glass slide. A solid surface on which DNA molecules are amplified typically comprise a plurality of first and second bound oligonucleotides, the first complementary to a sequence near or at one end of a target polynucleotide and the second complementary to a sequence near or at the other end of a target polynucleotide. This arrangement permits bridge amplification, such as described in US20140121116. The DNA molecules are then annealed to a sequencing primer and sequenced in parallel base-by-base using a reversible terminator approach. Hybridization of a sequencing primer may be preceded by cleavage of one strand of a double-stranded bridge polynucleotide at a cleavage site in one of the bound oligonucleotides anchoring the bridge, thus leaving one single strand not bound to the solid substrate that may be removed by denaturing, and the other strand bound and available for hybridization to a sequencing primer. Typically, the Illumina Genome Analyzer System utilizes flow-cells with 8 channels, generating sequencing reads of 18 to 36 bases in length, generating>1.3 Gbp of high-quality data per run (see www. illumina. com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β, γ, or other terminal phosphate group on a nucleoside polyphosphate, the label is not incorporated into the nascent strand, and instead, natural DNA is produced. Observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,917,726, 7,033,764, 7,052,847, 7,056,676, 7,170,050, 7,361,466, and 7,416,844; and US 20070134128).

In some embodiments, the nucleic acids in the sample can be sequenced by ligation. This method typically uses a DNA ligase enzyme to identify the target sequence, for example, as used in the polony method and in the SOLID technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

According to some embodiments, a sequence difference between sequencing reads and a reference sequence are called as a genuine sequence variant (e.g., existing in the sample prior to amplification or sequencing, and not a result of either of these processes) if it occurs in both strands of the double stranded nucleic acid molecule. Because sequence variants that are the result of amplification or sequencing errors are unlikely to be duplicated exactly (e.g., position and type) on two different strands comprising complements of the same target sequence, adding this validation parameter greatly reduces the background of erroneous sequence variants, with a concurrent increase in the sensitivity and accuracy of detecting actual sequence variation in a sample. In some embodiments, a sequence variant having a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower is sufficiently above background to permit an accurate call. In some embodiments, the sequence variant occurs with a frequency of about or less than about 0.1%. In some embodiments, the frequency of a sequence variant is sufficiently above background when such frequency is statistically significantly above the background error rate (e.g., with a p-value of about or less than about 0.05, 0.01, 0.001, 0.0001, or lower). In some embodiments, the frequency of a sequence variant is sufficiently above background when such frequency is about or at least about 2-fold, 3-fold, 4-fold, 5-fold, 6-fold, 7-fold, 8-fold, 9-fold, 10-fold, 25-fold, 50-fold, 100-fold, or more above the background error rate (e.g., at least 5-fold higher). In some embodiments, the background error rate in accurately determining the sequence at a given position is about or less than about 1%, 0.5%, 0.1%, 0.05%, 0.01%, 0.005%, 0.001%, 0.0005%, or lower. In some embodiments, the error rate is lower than 0.001%.

In some embodiments, identifying a genuine sequence variant (also referred to as “calling” or “making a call”) comprises optimally aligning one or more sequencing reads with a reference sequence to identify differences between the two, as well as to identify strands of the double stranded nucleic acid molecule. In general, alignment involves placing one sequence along another sequence, iteratively introducing gaps along each sequence, scoring how well the two sequences match, and preferably repeating for various positions along the reference. The best-scoring match is deemed to be the alignment and represents an inference about the degree of relationship between the sequences. In some embodiments, a reference sequence to which sequencing reads are compared is a reference genome, such as the genome of a member of the same species as the subject. A reference genome may be complete or incomplete. In some embodiments, a reference genome consists only of regions containing target polynucleotides, such as from a reference genome or from a consensus generated from sequencing reads under analysis. In some embodiments, the reference sequence consists of only a portion of a reference genome, such as regions corresponding to one or more target sequences under analysis (e.g., one or more genes, or portions thereof). In some embodiments, sequencing reads are aligned to multiple different reference sequences.

In a typical alignment, a base in a sequencing read alongside a non-matching base in the reference indicates that a substitution mutation has occurred at that point. Similarly, where one sequence includes a gap alongside a base in the other sequence, an insertion or deletion mutation (an “indel”) is inferred to have occurred. When it is desired to specify that one sequence is being aligned to one other, the alignment is sometimes called a pairwise alignment. Multiple sequence alignment generally refers to the alignment of two or more sequences, including, for example, by a series of pairwise alignments. In some embodiments, scoring an alignment involves setting values for the probabilities of substitutions and indels. When individual bases are aligned, a match or mismatch contributes to the alignment score by a substitution probability, which could be, for example, 1 for a match and 0.33 for a mismatch. An indel deducts from an alignment score by a gap penalty, which could be, for example, −1. Gap penalties and substitution probabilities can be based on empirical knowledge or a priori assumptions about how sequences mutate. Their values affect the resulting alignment. Examples of algorithms for performing alignments include, without limitation, the Smith-Waterman (SW) algorithm, the Needleman-Wunsch (NW) algorithm, algorithms based on the Burrows-Wheeler Transform (BWT), and hash function aligners such as Novoalign (Novocraft Technologies; available at www.novocraft.com), ELAND (Illumina, San Diego, Calif.), SOAP (available at soap.genomics.org.cn), and Maq (available at maq.sourceforge.net). One exemplary alignment program, which implements a BWT approach, is Burrows-Wheeler Aligner (BWA) available from the SourceForge web site maintained by Geeknet (Fairfax, Va.). BWT typically occupies 2 bits of memory per nucleotide, making it possible to index nucleotide sequences as long as 4G base pairs with a typical desktop or laptop computer. The pre-processing includes the construction of BWT (i.e., indexing the reference) and the supporting auxiliary data structures. BWA includes two different algorithms, both based on BWT. Alignment by BWA can proceed using the algorithm bwa-short, designed for short queries up to about 200 by with low error rate (<3%) (Li H. and Durbin R. Bioinformatics, 25:1754-60 (2009)). The second algorithm, BWA-SW, is designed for long reads with more errors (Li H. and Durbin R. (2010). Fast and accurate long-read alignment with Burrows-Wheeler Transform. Bioinformatics, Epub.). The bwa-sw aligner is sometimes referred to as “bwa-long”, “bwa long algorithm”, or similar. An alignment program that implements a version of the Smith-Waterman algorithm is MUMmer, available from the SourceForge web site maintained by Geeknet (Fairfax, Va.).

MUMmer is a system for rapidly aligning entire genomes, whether in complete or draft form (Kurtz, S., et al., Genome Biology, 5: R12 (2004); Delcher, A. L., et al., Nucl. Acids Res., 27:11 (1999)). For example, MUMmer 3.0 can find all 20-basepair or longer exact matches between a pair of 5-megabase genomes in 13.7 seconds, using 78 MB of memory, on a 2.4 GHz Linux desktop computer. MUMmer can also align incomplete genomes; it can easily handle the 100s or 1000s of contigs from a shotgun sequencing project and will align them to another set of contigs or a genome using the NUCmer program included with the system. Other non-limiting examples of alignment programs include: BLAT from Kent Informatics (Santa Cruz, Calif.) (Kent, W. J., Genome Research 4:656-664 (2002)); SOAP2, from Beijing Genomics Institute (Beijing, Conn.) or BGI Americas Corporation (Cambridge, Mass.); Bowtie (Langmead, et al., Genome Biology, 10:R25 (2009)); Efficient Large-Scale Alignment of Nucleotide Databases (ELAND) or the ELANDv2 component of the Consensus Assessment of Sequence and Variation (CASAVA) software (Illumina, San Diego, Calif.); RTG Investigator from Real Time Genomics, Inc. (San Francisco, Calif.); Novoalign from Novocraft (Selangor, Malaysia); Exonerate, European Bioinformatics Institute (Hinxton, UK) (Slater, G., and Birney, E., BMC Bioinformatics 6:31(2005)), Clustal Omega, from University College Dublin (Dublin, Ireland) (Sievers F., et al., Mol Syst Biol 7, article 539 (2011)); ClustalW or ClustalX from University College Dublin (Dublin, Ireland) (Larkin M. A., et al., Bioinformatics, 23, 2947-2948 (2007)); and FASTA, European Bioinformatics Institute (Hinxton, UK) (Pearson W. R., et al., PNAS 85(8):2444-8 (1988); Lipman, D. J., Science 227(4693):1435-41 (1985)).

Typically, the sequencing data is acquired from large scale, parallel sequencing reactions. Many of the next generation high-throughput sequencing systems export data as FASTQ files, although other formats may be used. In some embodiments, sequences are analyzed to identify repeat unit length (e.g., the monomer length), the junction formed by ligation, and any true variation with respect to a reference sequence, typically through sequence alignment. Identifying the repeat unit length can include computing the regions of the repeated units, finding the reference loci of the sequences (e.g., when one or more sequences are particularly targeted for amplification, enrichment, and/or sequencing), the boundaries of each repeated region, and/or the number of repeats within each sequencing run. Sequence analysis includes analyzing sequence data for both strands of a duplex. As noted above, in some embodiments, an identical variant that appears the sequences of reads from different strands of the double stranded nucleic acid molecule is considered a confirmed variant. In some embodiments, a sequence variant may also be considered a confirmed, or genuine, variant if it occurs in more than one repeated unit of the same polynucleotide, as the same sequence variation is likewise unlikely to occur at the same position in a repeated target sequence within the same concatemer. The quality score of a sequence may be considered in identifying variants and confirmed variants, for example, the sequence and bases with quality scores lower than a threshold may be filtered out. Other bioinformatics methods can be used to further increase the sensitivity and specificity of the variant calls.

In some embodiments, statistical analyses may be applied to determination of variants (mutations) and quantitate the ratio of the variant in total DNA samples. Total measurement of a particular base can be calculated using the sequencing data. For example, from the alignment results calculated in previous steps, one can calculate the number of “effective reads,” that is, number of confirmed reads for each locus. The allele frequency of a variant can be normalized by the effective read count for the locus. The overall noise level, that is the average rate of observed variants across all loci, can be computed. The frequency of a variant and the overall noise level, combined with other factors, can be used to determine the confidence interval of the variant call. Statistical models such as Poisson distributions can be used to assess the confidence interval of the variant calls. The allele frequency of variants can also be used as an indicator of the relative quantity of the variant in the total sample.

### Computer Systems

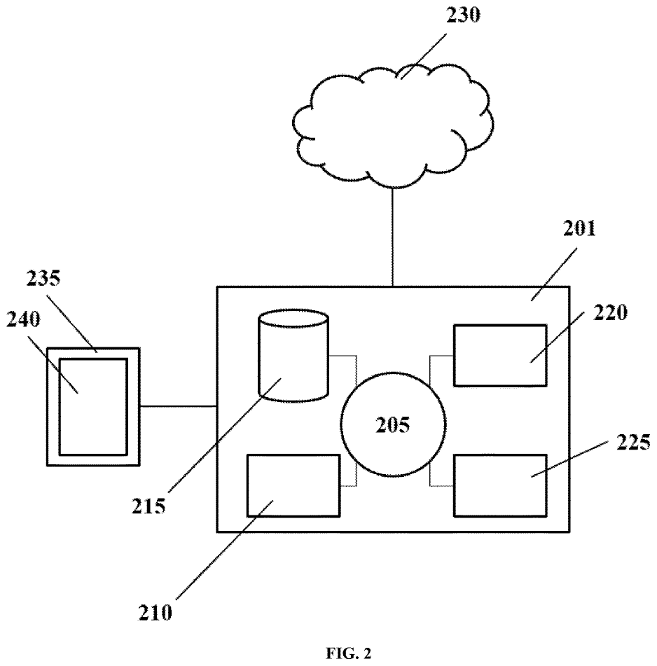

The present disclosure provides computer systems that are programmed to implement methods of the disclosure. FIG. 2 shows a computer system 201 that is programmed or otherwise configured to process nucleic acid molecules according to methods provided herein. The computer system 201 can regulate various aspects of nucleic acid processing methods of the present disclosure, such as, for example, providing a reaction mixture comprising a plurality of double stranded nucleic acid molecules and a plurality of oligonucleotides wherein, at least one of the plurality of oligonucleotides comprises a 5′ end and a 3′ end that are capable of base pairing with each other to yield a stem loop structure in the reaction mixture; subjecting the reaction mixture to a ligation reaction to ligate a first oligonucleotide of the plurality of oligonucleotides to a 5′ end of a double stranded nucleic acid molecule of the plurality of double stranded nucleic acid molecules and a second oligonucleotide of the plurality of oligonucleotides to a 3′ end of the double stranded nucleic acid molecule of the plurality of double stranded nucleic acid molecules, thereby creating at least one nucleic acid molecule without an exposed 5′ or 3′ end; modifying at least one nucleotide of the nucleic acid molecule to create a mismatched base pair within the nucleic acid molecule to yield a modified nucleic acid molecule; subjecting the modified nucleic acid molecule or derivative thereof to amplification with a primer to create a linear product comprising one or more copies of the modified nucleic acid molecule; and sequencing the linear product or a derivative thereof to obtain a sequence of a first strand and a second strand of the double stranded nucleic acid molecule. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries, and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., processing nucleic acid molecules according to methods herein). Examples of remote computer systems include personal computers (e.g., portable PC), slate or tablet PC's (e.g., Apple® iPad, Samsung Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, processing nucleic acid molecules according to methods herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, facilitate methods of nucleic acid molecule processing.

While preferred embodiments of the present invention have been shown and described herein, it will be obvious to those skilled in the art that such embodiments are provided by way of example only. It is not intended that the invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Method for Duplex Sequencing

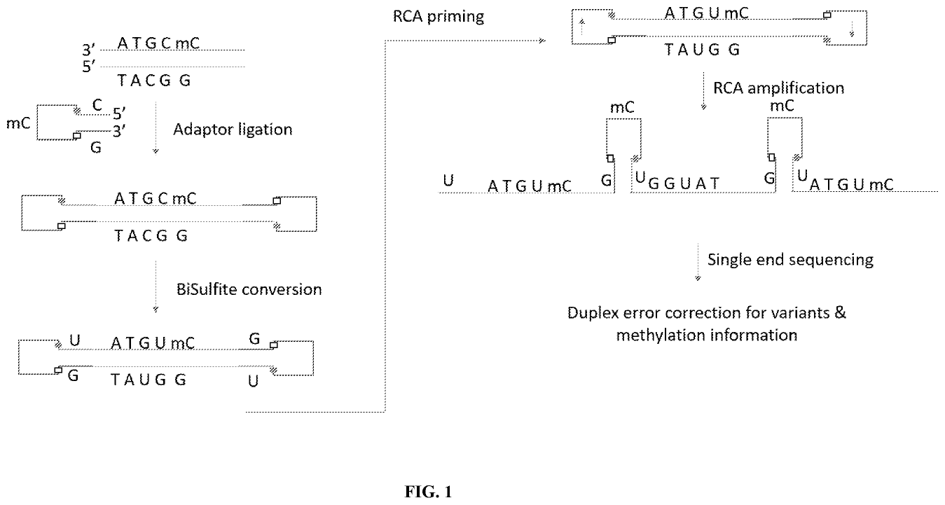

A plurality of double stranded nucleic acids is obtained from a biological sample from a subject. Adaptors are obtained where each adaptor has a 5′ end and a 3′ end that are complementary such that the ends base pair with each other and form a stem loop structure. Two of these adaptors are ligated to each end of the double stranded nucleic acids to form a nucleic acid molecule that has no exposed 5′ or 3′ ends. This nucleic acid molecule is subjected to bisulfite conversion which converts unmethylated cytosines in the nucleic acid molecule to uracil. This modified molecule is then subjected to rolling circle amplification using primers that bind to the adaptors. This creates a linear product that has a copy of each strand of the double stranded nucleic acid molecule separated by a copy of each adaptor. These amplification products are sequenced, and any identified variants are subjected to error correction based on the sequence of the complementary strand from the double stranded nucleic acid molecule. This method is summarized in FIG. 1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of processing a nucleic acid comprising:
(a) providing a reaction mixture comprising: (i) a plurality of double stranded nucleic acid molecules,
(ii) a plurality of oligonucleotides, wherein at least one of said plurality of oligonucleotides comprises a 5′ end and a 3′ end that are capable of base pairing with each other to yield a stem loop structure in said reaction mixture;
(b) subjecting said reaction mixture to a ligation reaction to ligate (i) a first oligonucleotide of said plurality of oligonucleotides to a 5′ end of a double stranded nucleic acid molecule of said plurality of double stranded nucleic acid molecules and (ii) a second oligonucleotide of said plurality of oligonucleotides to a 3′ end of said double stranded nucleic acid molecule of said plurality of double stranded nucleic acid molecules, thereby creating at least one nucleic acid molecule without an exposed 5′ or 3′ end;
(c) subjecting said nucleic acid molecule or derivative thereof to amplification with a primer to create a linear product comprising one or more copies of said nucleic acid molecule; and
(d) sequencing said linear product or a derivative thereof to obtain a sequence of a first strand and a second strand of said double stranded nucleic acid molecule.

2. The method of claim 1, wherein said first oligonucleotide or said second oligonucleotide comprises a methylated nucleotide.

3. The method of claim 1 or claim 2, wherein said double stranded nucleic acid molecule comprises a methylated nucleotide.

4. The method of claim 1 or claim 2, wherein said first oligonucleotide or said second oligonucleotide comprises one or more methylated cytosines.

5. The method of claim 2 or claim 3, wherein said methylated nucleotide comprises methylated cytosine or methylated adenine.

6. The method of any one of claims 1 to 5, wherein said polymerase is a strand displacing polymerase.

7. The method of any one of claims 1 to 6, wherein subsequent to (b), modifying at least one nucleotide of said nucleic acid molecule to create a mismatched base pair within said nucleic acid molecule to yield a modified nucleic acid molecule.

8. The method of claim 7, wherein said modifying comprises a chemical or enzymatic modification that converts a methylated cytosine to a uracil or an unmethylated cytosine to a uracil.

9. The method of any one of claims 1 to 8, wherein said amplification product is a concatemer.

10. The method of any one of claims 1 to 9, further comprising processing said sequence to identify a sequence variant when a sequence difference is present in said sequence of said first strand and said sequence of said second strand.

11. The method of claim 10, wherein said sequence variant comprises a single nucleotide variant, a fusion, an insertion, or a deletion.

12. The method of any one of claims 1 to 10, further comprising processing said sequence to identify an epigenetic modification when an epigenetic difference is present in said sequence of said first strand and confirmed by said sequence of said second strand.

13. The method of any one of claims 1 to 12, wherein said primer is a random primer.

14. The method of any one of claims 1 to 12, wherein said primer is a target specific primer.

15. The method of any one of claims 1 to 14, wherein said primer binds to a sequence of said first oligonucleotide or said second oligonucleotide.

16. The method of any one of claims 1 to 14, wherein said primer binds to a sequence of said first strand or said second strand of said double stranded nucleic acid molecule.

17. The method of any one of claims 1 to 16, wherein said plurality of double stranded nucleic acid molecules comprise double stranded deoxyribonucleic acid, double stranded ribonucleic acid, or a combination thereof.

18. The method of any one of claims 1 to 17, wherein said plurality of double stranded nucleic acid molecules is derived from a cell-free nucleic acid sample.

19. The method of any one of claims 1 to 18, wherein sequencing comprises (i) bringing said plurality of sheared polynucleotides or a derivative thereof in contact with a plurality of nucleotides in the presence of a polymerase to incorporate one or more nucleotides of said plurality of nucleotides into a growing strand complementary to a strand of said sheared polynucleotides or derivative thereof, and (ii) detecting one or more signals indicative of incorporation of said one or more nucleotides into said growing strand.

20. The method of any one of claims 1 to 19, wherein said sequencing comprises sequencing by ligation.

21. The method of any one of claims 1 to 20, wherein said plurality of double stranded nucleic acid molecules is derived from a bodily fluid.

22. The method of claim 21, wherein said bodily fluid comprises urine, saliva, blood, serum, sweat, or plasma.

23. The method of any one of claims 1 to 22, wherein said plurality of double stranded nucleic acids is derived from a biological sample of a subject, which biological sample comprises blood, saliva, serum, plasma, urine, menstrual fluid, sweat, skin, bone marrow, lung tissue, liver tissue, pancreatic tissue, stomach tissue, intestinal tissue, kidney tissue, bladder tissue, brain tissue, spinal cord tissue, tongue tissue, larynx tissue, esophageal tissue, spleen tissue, thymus tissue, thyroid tissue, breast tissue, prostate tissue, testicular tissue, or a tumor thereof.

24. The method of any one of claims 1 to 23, wherein said plurality of double stranded nucleic acid molecules is derived from a tumor sample of a subject.
